(19) United States
(12) Patent Application Publication
(10) Pub. No.: US 2020/0179633 A1
(43) Pub. Date: Jun. 11, 2020

(54) CPAP MASKS CONTAINER

(71) Applicant: Craig Beasley, Ruther Glen, VA (US)

(72) Inventor: Craig Beasley, Ruther Glen, VA (US)

(21) Appl. No.: 16/211,129

(22) Filed: Dec. 5, 2018

(51) Int. Cl. A61M 16/06; A61M 16/00; A61M 16/08

(52) U.S. Cl. CPC A61M 16/06; A61M 2209/06; A61M 16/0875; A61M 16/0003

(57) ABSTRACT

A system and method for preventing germs, dust, and debris from being collected and inhaled by a user of a CPAP mask is presented. The user places his/her CPAP face mask into a storage case. The storage case has a connector for a hose used by the CPAP face mask when connecting to a CPAP machine. The hose is connected to the storage case and the other end of the hose has an end cap. A machine cap is further presented to place on the intake/output of the CPAP machine. The case has a housing, the case could be a storage case. There is at least one end cap that can be securely attached to a connecting port of the case. A second end cap for the end of the hose and a cap for the CPAP machine.

### FIELD OF THE INVENTION

This invention relates to CPAP masks. More particularly, it relates to safe storage and protection of CPAP masks and air hoses.

### BACKGROUND

Obstructive Sleep Apnea (OSA) is intermittent blockage of a person's airflow while they sleep due to movement of their tongue or other soft tissue. Such blockages can happen hundreds of times each night, causing poor sleep and oxygen deprivation. Basically, the person temporarily stops breathing during each blockage. Unfortunately, the person might not even be aware of this serious problem because they never fully wake up during blockages.

Obstructive sleep apnea can cause serious long-term harmful effects. These harmful effects include: disrupted sleep; chronic fatigue; morning headaches; irritability; brain damage; cognitive dysfunction; impotency; high blood pressure; heart attacks; congestive heart failure; motor vehicle crashes; job-site accidents; and even death. Despite these harmful effects, it is estimated that only 5% to 8% of the affected population are treated. Approximately 20 million Americans and 35 million people worldwide have obstructive sleep apnea and the number is growing rapidly.

The first-line therapy for most people who are diagnosed with obstructive sleep apnea is Continuous Positive Airway Pressure (CPAP). CPAP keeps the airway open with a stream of pressurized air that is channeled into a person's nose (or nose and mouth) while they sleep. The positive pressure keeps their tongue and other soft tissue from blocking the airway. A CPAP machine continuously pumps pressurized air into a CPAP mask that fits over a person's face while they sleep. Some CPAP masks cover only the sleeper's nose. Other CPAP masks cover both the sleeper's nose and mouth.

In many respects, the mask is the weak link of CPAP. CPAP only works if a person tolerates wearing the CPAP mask and if the mask does not leak pressurized air. If the mask fits too tightly in some places where the mask presses against the person's face, then this can cause skin irritation, red marks, and pain. These problems contribute to high non-compliance with CPAP therapy; many people are not willing to wear a CPAP mask while they sleep. Estimates of the percentage of people who should wear CPAP masks but do not wear them range as high as 50%. On the other hand, if the mask fits too loosely and there are gaps in some places between the mask and the person's face, then the mask leaks pressurized air. Air leaks erode the clinical effectiveness, of CPAP.

One of the main challenges for CPAP mask owners is the care and safety of the CPAP mask when the owner is not using the CPAP mask. The storage of CPAP masks has not been solved to prevent damage or uncleanliness of the CPAP masks. Another challenge is the care of the air hose to be used with the CPAP mask and it storage such that when the owner has need to use the CPAP mask that the air hose is also available.

### BRIEF DESCRIPTION OF THE DRAWINGS

FIG. 1 is an illustrated CPAP machine and tools are presented.

FIG. 2 is an illustrated exemplary connected charging storage case.

FIG. 3 is an illustrated cap for the storage case of FIG. 2.

FIG. 4 is an illustrated plug for the CPAP machine of FIG. 2.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

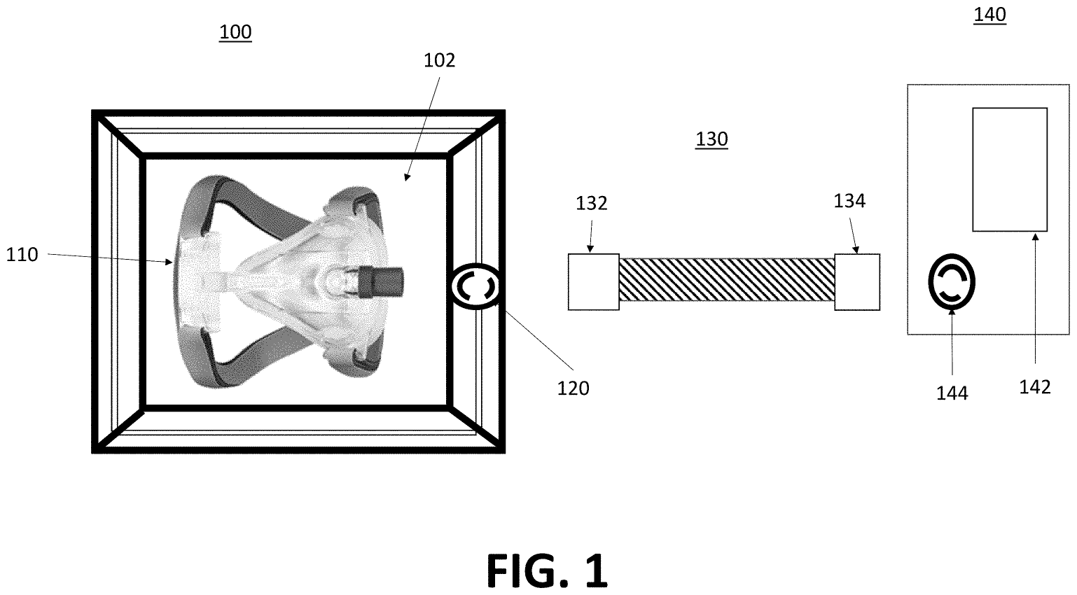

Referring to FIG. 1, an illustrated view of the tools and CPAP machine are presented. Continuous positive airway pressure (CPAP) is an effective treatment for moderate to severe obstructive sleep apnea.

Patients with obstructive sleep apnea treated with CPAP wear a face mask during sleep which is connected to a pump (CPAP machine) that forces air into the nasal passages at pressures high enough to overcome obstructions in the airway and stimulate normal breathing. The airway pressure delivered into the upper airway k continuous during both inspiration and expiration.

Nasal CPAP is currently the preferred treatment for moderate to severe obstructive sleep apnea. CPAP is safe and effective, even in children. Daytime sleepiness improves or resolves. Heart function and hypertension also improve. And, importantly, the quality of life improves.

A housing 100 has an internal cavity 102. The housing 100 may function as a storage case, etc. The housing 100 is preferably made of plastic but may be any type of material contemplated. The housing 100 is useful for preventing germs, dust, and debris from being collected and inhaled by a user. The housing 100 is preferably 2 feet in height, seven (7) inches long and seven (7) inches wide, but may be adjusted to be any height, length, and width one of ordinary skill in the art may contemplate. The housing 100 is preferably square but may be other shapes as one of skill in the art may determine.

The internal cavity 102 is of such a size to fit articles or tools associated with CPAP users or other type of devices. The housing 100 has a receiving port 120. The receiving port 120 receives a hose such as hose 132.

The housing 100 as shown contains a facemask 110 in the internal cavity 102. The facemask 110 is useful when a user desires or needs to use a CPAP machine. The facemask 100 is generally removed from the internal cavity 102 of the housing 100. The facemask 110 is coupled to the hose 132 and the hose 132 is coupled further to a CPAP machine 140. The user then secures the facemask 110 over their mouth and nose. This provides support for breathing issues such as apnea when the user is sleeping.

The hose 132 has a first end 132 and a second end 134. The first end 132 is generally coupled to the housing 100 when the facemask 110 is not in use. The first end 132 is coupled to the facemask 110 when the user desires or needs to use the CPAP machine 140. The second end 134 is coupled to the CPAP machine 140 when the user desires or needs to use the CPAP machine 140.

When the CPAP machine 140 is not in use and the facemask 110 is being stored in housing 100, the first end of the hose 130 is coupled to the receiving port 120 of the housing 100. The second end 134 would generally have a cap to keep germs, dirt, dust, etc. from entering the hose 130 and the facemask 110.

The CPAP machine 140 is coupled to the facemask 110 by the hose 130. The second end 134 is coupled to a egress port 144 of the CPAP machine 140. The CPAP machine 140 further has a controller 142 for displaying vital information, turning the CPAP machine 140 on and off, etc. The controller 142 may be a touchscreen, an LED display, etc.

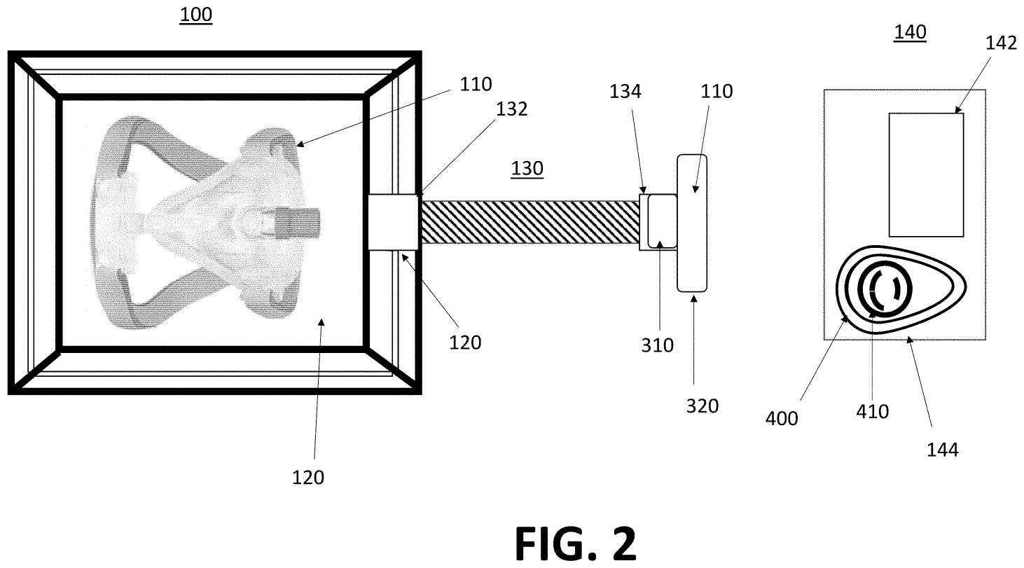

FIG. 2 shows the housing 100 being used to store the facemask 110. The facemask 110 is placed into internal cavity 102 of the housing 100. The first end 132 of the hose 130 is coupled to the receiving port 120 of the housing 100. The second end 134 of the hose is disconnected from the CPAP machine 140 and a endcap 300 engages the second end 134 of the hose 130 to keep dirt, dust, etc. from entering the hose 130. The endcap 300 has a connector 310 and a stop 320. The connector 310 is securely inserted into the second end 134 of the hose 130 and is pushed until the stop 320 is flush against the second end 134 of the hose 130.

A plug 400 is coupled to the egress port 144 of the CPAP machine 140 to keep dirt, dust, etc. from entering or clogging the egress port 144 of the CPAP machine 140. The plug 400 has a cover 410 which is shown as clear in FIG. 2 but may be art work, colored, etc.

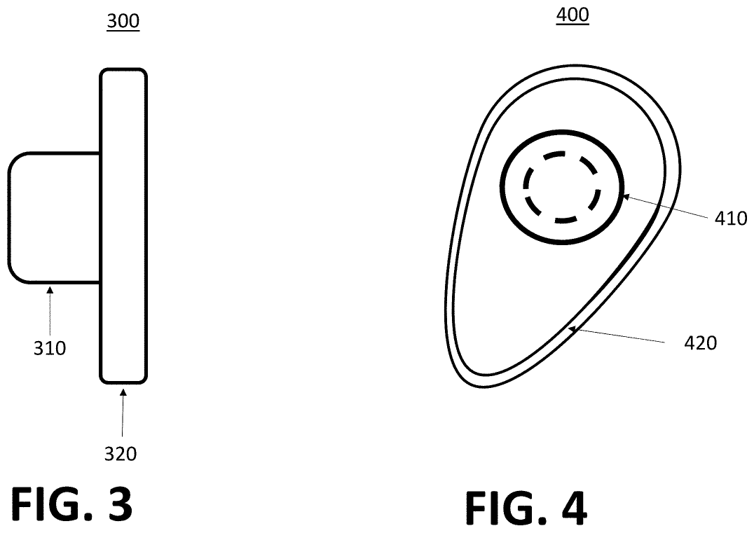

Moving now to FIG. 3, an endcap 300 is presented. Endcap 300 is useful to insert securely into the first end 132 and/or the second end 134 of the hose 130 to help keep dust, dirt, etc. from entering and contaminating the hose 130.

The endcap 300 may be one or more endcaps 300. Each of the one or more endcaps may be the same or different sizes, preferably the one or more endcaps 300 are of such a size that will fit securely into the first end 132 or second end 134 of the hose 130. The one or more endcaps are preferably made of plastic, but one of skill in the art may use different materials. Each of the one or more endcaps 300 may be made of the same material or of different materials.

The one or more endcaps 300 has a connector 310 and a stop 320. The connector 310 of the endcap 300 is preferably twenty-two (22) millimeters in diameter but may be of any size to fit securely into the end of a hose being used. The stop 320 is preferably of a greater size than the connector 310. The stop 320 provides an end point for the endcap 300 to be inserted into the first end 132 or second end 134 of the hose 130.

In FIG. 4, a plug 400 is presented. The plug 400 is generally used to securely prevent the egress port 144 of the CPAP machine 140 from having contaminants enter, such as dirt, dust, etc. The plug 400 is preferably twenty-two (22) millimeters in diameter but may be of any size to fit securely into the egress port 144 of the CPAP machine 140. The plug 400 is preferably made of plastic, but one of skill in the art may use other materials as determined by the one of skill in the art.

The plug 400 has a cover 410. The cover 410 may be clear, have artwork, etc. The plug 400 alternatively has a further outer surface 420 which may be useful to help handling of the plug 400 and ease inserting the plug 400 into the egress port 144 of the CPAP machine 140 or to remove the plug 400 from the egress port 144 of the CPAP machine.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for preventing germs, dust, and debris from being collected and inhaled by a user, comprising the steps:
placing a face mask in a storage case;
connecting a hose to said storage case;
placing a hose end cap on an end of said hose; and
placing a machine end cap on an end of a CPAP machine.

2. A system for preventing germs, dust, and debris from being collected and inhaled by a user, comprising:
a housing having an internal cavity;
at least one end cap, said at least one end cap configured to couple to a first end of said housing;
a second end cap, said second end cap configured to couple to an end of a hose; and
at least one plug, said at least one plug configured to couple to an opening of a CPAP machine.

3. The system of claim 2, wherein said housing is a storage case.

4. The system of claim 2, wherein said housing houses a face mask.

5. The system of claim 2, wherein said housing is approximately 2 inches in height.

6. The system of claim 2, wherein said housing is approximately 7 inches in length.

7. The system of claim 2, wherein said housing is approximately 7 inches in width.

8. The system of claim 2, wherein said housing is made of a plastic material.

9. The system of claim 2, wherein said at least one end cap is made of a plastic material.

10. The system of claim 2, wherein said second end cap is made of a plastic material.

11. The system of claim 2, wherein at least one plug is made of a plastic material.

12. The system of claim 2, wherein said housing is square in shape.

13. The system of claim 2, wherein said at least one end cap is round in shape.

14. The system of claim 2, wherein said second end cap is round in shape.

15. The system of claim 2, wherein said at least one plug is round in shape.

16. The system of claim 2, wherein said at least one end cap has a diameter of approximately 22 mm.

17. The system of claim 2, wherein said at least one plug has a diameter of approximately 22 mm.
